(19) United States
(12) Patent Application Publication
(10) Pub. No.: US 2024/0335438 A1
(43) Pub. Date: Oct. 10, 2024

(54) TARGETING DOT1L AND SMARCA4/2 FOR THE TREATMENT OF MLLR LEUKEMIA

(71) Applicant: DANA-FARBER CANCER INSTITUTE, INC., Boston, MA (US)

(72) Inventors: Jun Qi, Sharon, MA (US); Lingling Dai, Boston, MA (US); Florian Perner, Boston, MA (US); Scott Armstrong, Wayland, MA (US)

(73) Assignee: DANA-FARBER CANCER INSTITUTE, INC., Boston, MA (US)

(21) Appl. No.: 18/580,030

(22) PCT Filed: Jul. 18, 2022
(86) PCT No.: PCT/US2022/073835

§ 371 Date: Jan. 17, 2024

Related U.S. Application Data
(60) Provisional application No. 63/223,302, filed on Jul. 19, 2021.

(51) Int. Cl. A61K 31/454; A61K 31/52; A61K 31/53; A61P 35/02

(52) U.S. Cl. CPC A61K 31/454; A61K 31/52; A61K 31/53; A61P 35/02

(57) ABSTRACT

Disclosed is a method of treating cancer with a compound that possesses degradation activity against SMARCA4/2 in combination with a DOT1L r inhibitor.

### RELATED APPLICATIONS

This application claims the benefit of priority under 35 U.S.C. § 119 (e) to U.S. Provisional Application No. 63/223,302, filed Jul. 19, 2021, which is incorporated herein by reference in its entirety.

### GOVERNMENT LICENSE RIGHTS

This invention was made with government support under grant number R01CA176745 awarded by the National Institutes of Health. The government has certain rights in the invention.

### BACKGROUND OF THE INVENTION

The mixed lineage leukemia (MLL; official symbol KMT2A) is one of the most frequently translocated gene in hematologic malignancies, and is present in more than 80% of infant leukemias and up to 10% of adult acute myeloid leukemias. MLL rearrangements (MLLr) lead to loss of the C-terminal part including the catalytic SET domain and replacement of variety of fusion proteins (MLL-FPs), most commonly AF4, AF9 and ENL. Despite recent progress in the understanding of MLLr leukemias, the disease remains difficult to cure, and thus remains as a significant unmet medical need.

Brahma-related gene 1 (BRG1/SMARCA4) and Brahma (BRM/SMARCA2) are two crucial components of the SWItch/Sucrose Non-Fermentable (SWI/SNF) complex. At the protein level, they share approximately 75% identity and both proteins belong to the SNF2 family of chromatin-remodeling proteins. (Epigenetics and Dermatology, Lu Q., Chang C. C. and Richardson B. C. (eds.), Academic Press, Cambridge, MA (2015)). They directly participate in DNA replication, repair, and recombination through modifying chromatin or recruiting relevant proteins (Hodges, C. et al., Cold Spring Harb. Perspect. Med. 6:a026930 (2016)). Mutations in SWI/SNF subunits have been seen in more than 20% of all human cancers, highlighting their critical roles in tumorigenesis (Kadoch, et al., Nat. Genet. 45:592-601 (2013)).

Genetic studies have demonstrated that MLL-fusion proteins (FPs) gain the function to recruit coactivators including disruptor of telomeric silencing 1-like protein (DOT1L), the only known H3K79 methyltransferase, and super elongation complex (SEC) to target gene loci and induce abnormal transcription of these genes. Many studies have demonstrated that DOT1L is required for the development and maintenance of MLLr leukemias. Consequently, much effort has been devoted to the development of small molecules to inhibit DOT1L catalytic functions. The recent development of DOT1L inhibitor, EPZ-5676 (pinometostat), showed remarkably selective anti-proliferative effects on MLL rearranged cells, and has been progressed in phase I and II clinical trials. Despite its promising side effect profile for the treatment of MLLr acute myeloid leukemia (AML), the low effective responses and subsequent relapses as a monotherapy have limited its further development. Moreover, the exact mechanisms by which DOT1L and H3K79 methylation modulate local structure of chromatin and facilitate the binding of chromatin regulators in MLLr leukemia remain elusive. The apparent disconnect between substantial reduction of global H3K79 dimethylation and less marked downstream pharmacodynamic responses, namely target gene suppression and leukemic cell death or differentiation, underlines the need for extreme target inhibition to achieve the desired functional consequences (Stauffer, F. et al., ACS Med. Chem. Lett. 10:1655-1660 (2019)).

### SUMMARY OF THE INVENTION

The present invention includes methods for targeting DOT1L and SMARCA4/2 to treat cancer.

A first aspect of the present invention is directed to a method of treating cancer wherein cells of the cancer express SMARCA4/2 and DOT1L (and/or the cancer has SMARCA4/2 and DOT1L activity), comprising co-administering a therapeutically effective amount of compound (1):

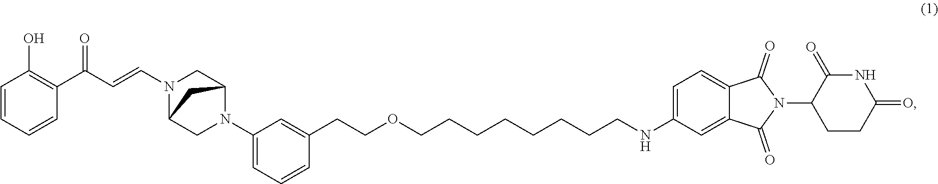 [SMILES: O=C1CCC(N2C(=O)c3ccc(NCCCCCCCCOCCc4cccc(N5C[C@H]6C[C@@H]5CN6/C=C/C(=O)c5ccccc5O)c4)cc3C2=O)C(=O)N1]

or a pharmaceutically acceptable salt thereof, and a therapeutically effective amount of a DOT1L inhibitor, or a pharmaceutically acceptable salt thereof, to a subject in need thereof. Embodiments of this aspect of the invention may achieve a synergistic anti-cancer effect.

In some embodiments, the DOT1L inhibitor is EPZ-5676. In some embodiments, the DOT1L inhibitor is Dot1L-IN-4. In some embodiments, the DOT1L inhibitor is Dot1L-IN-5.

The methods of the present disclosure may provide beneficial effects in terms of enhancing anti-cancer activity while improving the tolerability of SMARCA4/2 inactivation.

### BRIEF DESCRIPTION OF THE DRAWINGS

FIG. 1A-FIG. 1B are a series of graphs showing that S31 binds to SMARCA4/2 and CRBN.

FIG. 2 is a Western Blot showing that S31 degraded SMARCA4/2 in leukemia cells.

FIG. 3 is a graph showing that S31 induces cytotoxicity in leukemia cells.

FIG. 4A-FIG. 4C are a series of graphs and schematics showing that BRM14 and EPZ5676 leads to synthetic lethality with long exposure. FIG. 4A shows the chemical structure of BRM 14. FIG. 4B is a schematic for combination treatment of BRM14 and EPZ5676. FIG. 4C is a series of graphs showing that BRM14 and EPZ5676 leads to synthetic lethality in MOLM13 cells.

FIG. 5A-FIG. 5B are a series of graphs and schematics showing that S31 and EPZ5676 shows a synergistic effect in treatment. FIG. 5A is a schematic for combination treatment of S31 and EPZ5676. FIG. 5B is a series of graphs showing that S31 and EPZ5676 has a synergistic effect in MOLM13 cells.

FIG. 6 is a series of bar graphs showing that synthetic lethality was observed in MLL-fusion cells.

FIG. 7A-FIG. 7B are a series of graphs and schematics showing that S31 and EPZ5676 leads to long-lasting synergistic effect. FIG. 7A is a schematic for combination treatment of S31 and EPZ5676. FIG. 7B is a series of graphs showing that S31 and EPZ5676 has a long-lasting synergistic effect in MOLM13 and MV4; 11 cells.

FIG. 8 is a schematic showing the protocol for evaluating efficacy of S31 and EPZ5676 ex vivo.

FIG. 9A is a series of flow cytometry images showing that S31 and EPZ5676 suppresses leukemia cell expansion. FIG. 9B is a graph showing that S31 and EPZ5676 suppresses leukemia cell expansion.

FIG. 10 is a Kaplan-Meier survival analysis graph showing that S31 and EPZ5676 improves the survival rates.

FIG. 11A is a heat map showing that S31 sensitizes MLL-AF9 targets to EPZ5676 in various cell lines. FIG. 11B is a gene set enrichment analysis (GSEA) showing that genes that directly bound by MLL-AF9 were significantly correlated with genes changed by S31 and EPZ5676 treatment. FIG. 11C is a bar graph showing that S31 and EPZ5676 have a synergistic effect in MYB and FLT3 cell lines.

FIG. 12A-FIG. 12B are a series of bar graphs showing that S31 and CMP10 leads to long-lasting synergistic effect. FIG. 12A is a bar graph showing that S31 and CMP10 (100 nM) has a long-lasting synergistic effect in MOLM13 cells. FIG. 12B is a bar graph showing that S31 and CMP10 (400 nM) has a long-lasting synergistic effect in MOLM13 cells.

FIG. 13A-FIG. 13B are a series of bar graphs showing that S31 and CMP11 leads to long-lasting synergistic effect. FIG. 13A is a bar graph showing that S31 and CMP11 (100 nM) has a long-lasting synergistic effect in MOLM13 cells. FIG. 13B is a bar graph showing that S31 and CMP11 (400 nM) has a long-lasting synergistic effect in MOLM13 cells.

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Therefore, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2%, or 1%) of the particular value modified by the term “about.”

The transitional term “comprising.” which is synonymous with “including.” “containing.” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Methods of the present invention utilize compound (1):

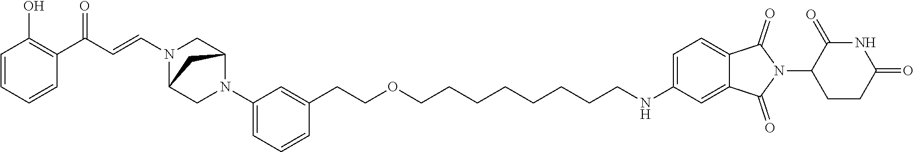 [SMILES: O=C1CCC(N2C(=O)c3ccc(NCCCCCCCCOCCc4cccc(N5C[C@H]6C[C@@H]5CN6/C=C/C(=O)c5ccccc5O)c4)cc3C2=O)C(=O)N1]

2-(2,6-dioxopiperidin-3-yl)-5-((8-(3-((1R,4R)-5-((E)-3-(2-hydroxyphenyl)-3-oxoprop-1-en-1-yl)-2,5-diazabicyclo[2.2.1]heptan-2-yl)phenethoxy)octyl)amino)isoindoline-1,3-dione (1, S31), and a DOT1L inhibitor or a pharmaceutically acceptable salt thereof. Compound (1) may be synthesized in accordance with known procedures (WO2020/264172).

Representative examples of DOT1L inhibitors that may be suitable for use in the present invention are known in the art. See, e.g., U.S. Patent Application Publication No. 2022/0184195 and International Patent Application Publication No. WO 2012/075381, each of which is incorporated herein by reference in its entirety.

In some embodiments, the DOT1L inhibitor is (2R,3R,4S,5R)-2-(6-amino-9H-purin-9-yl)-5-((((1s,3R)-3-(2-(5-(tert-butyl)-1H-benzo[d]imidazol-2-yl)ethyl)cyclobutyl)(isopropyl)amino)methyl)tetrahydrofuran-3,4-diol:

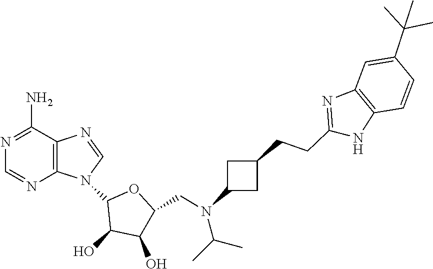

(“EPZ5676”). In some embodiments, the DOT1L inhibitor is(S)—N¹-((3-chloropyridin-2-yl)(2,2-difluorobenzo[d][1,3]dioxol-4-yl)methyl)-N²-(4-methoxy-6-(piperazin-1-yl)-1,3,5-triazin-2-yl)-4-(methylsulfonyl)benzene-1,2-diamine,

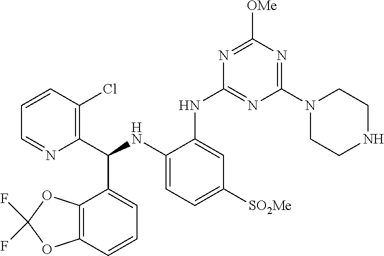 [SMILES: COc1nc(Nc2cc(S(C)(=O)=O)ccc2N[C@@H](c2cccc3c2OC(F)(F)O3)c2ncccc2Cl)nc(N2CCNCC2)n1]

(“CMP10”, “Dot1L-IN-4”). In some embodiments, the DOT1L inhibitor is (S)-3-((4-amino-6-methoxy-1,3,5-triazin-2-yl)amino)-4-(((3-chloropyridin-2-yl)(2,2-difluorobenzo[d][1,3]dioxol-4-yl)methyl)amino)benzenesulfonamide,

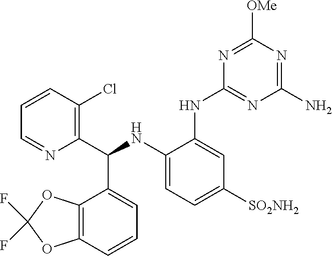 [SMILES: COc1nc(N)nc(Nc2cc(S(N)(=O)=O)ccc2N[C@@H](c2cccc3c2OC(F)(F)O3)c2ncccc2Cl)n1]

(“CMP11”, “Dot1L-IN-5”). DOT1L inhibitors, e.g., EPZ-5676, Dot1L-IN-4, and Dot1L-IN-5, are commercially available (MedChemExpress, Monmouth Junction. NJ).

Compound (1) and the DOT1L inhibitor may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or a base. Examples of pharmaceutically acceptable salts of the compound of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. The compound of the invention can form pharmaceutically acceptable salts with various organic bases such as lysine, arginine, guanidine, diethanolamine or metformin.

### Pharmaceutical Compositions

Administration of compound (1) and the DOT1L inhibitor may be facilitated by formulating them with a pharmaceutically acceptable carrier. The term “pharmaceutically acceptable carrier,” as known in the art, refers to a pharmaceutically acceptable material, composition or vehicle, suitable for administering the compound and DOT1L inhibtor to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound and DOT1L inhibitor from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients.

Broadly, compound (1) and the DOT1L inhibitor and their pharmaceutically acceptable salts may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound and DOT1L inhibitor may be administered relatively quickly such as in the case of a single-dose treatment and/or an acute condition.

In some embodiments, compound (1) and the DOT1L inhibitor are formulated for oral or intravenous administration (e.g., systemic intravenous injection).

Accordingly, compound (1) and the DOT1L inhibitor may be formulated into solid compositions (e.g., powders, tablets, dispersible granules, capsules, cachets, and suppositories), liquid compositions (e.g., solutions in which the compound and DOT1L inhibitor are dissolved, suspensions in which solid particles of the compound and DOT1L inhibitor are dispersed, emulsions, and solutions containing liposomes, micelles, or nanoparticles, syrups and elixirs); semi-solid compositions (e.g., gels, suspensions and creams); and gases (e.g., propellants for aerosol compositions). The compound and DOT1L inhibitor may also be formulated for rapid, intermediate or extended release.

Solid dosage forms for oral administration include capsules, tablets, pills, powders, and granules. In such solid dosage forms, the active compound and DOT1L inhibitor are mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compound, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, compound (1) and the DOT1L inhibitor may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound and DOT1L inhibitor, the liquid dosage forms may contain an aqueous or non-aqueous carrier (depending upon the solubility of the compound and DOT1L inhibitor) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound and DOT1L inhibitor may be prolonged by slowing its absorption, which may be accomplished by the use of a liquid suspension or crystalline or amorphous material with poor water solubility. Prolonged absorption of the compound and DOT1L inhibitor from a parenterally administered formulation may also be accomplished by suspending the compound and DOT1L inhibitor in an oily vehicle.

In certain embodiments, compound (1) and the DOT1L inhibitor may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound and DOT1L inhibitor in a biodegradable polymer, e.g., polylactide-polyglycolides, poly(orthoesters) and poly(anhydrides). The rate of release of the compound and DOT1L inhibitor may be controlled by varying the ratio of compound and DOT1L inhibitor to polymer and the nature of the particular polymer employed. Depot injectable formulations are also prepared by entrapping the compound and DOT1L inhibitor in liposomes or microemulsions that are compatible with body tissues. Furthermore, in other embodiments, the compound and DOT1L inhibitor are delivered in a targeted drug delivery system, for example, in a liposome coated with organ-specific antibody. In such embodiments, the liposomes are targeted to and taken up selectively by the organ.

The compositions may be formulated for buccal or sublingual administration, examples of which include tablets, lozenges and gels.

Compound (1) and the DOT1L inhibitor may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and DOT1L inhibitor and a suitable powder base such as lactose or starch.

Compound (1) and the DOT1L inhibitor may be formulated for topical administration which as used herein, refers to administration intradermally by invention of the formulation to the epidermis. These types of compositions are typically in the form of ointments, pastes, creams, lotions, gels, solutions and sprays.

Representative examples of carriers useful in formulating the compound and DOT1L inhibitor for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40)-stearate.

In some embodiments, the topical formulations may also include an excipient, an example of which is a penetration enhancing agent. These agents are capable of transporting a pharmacologically active compound and DOT1L inhibitor through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soy bean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound and DOT1L inhibitor are formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compound and DOT1L inhibitor may be accomplished by means of an iontophoretic patch. Transdermal patches may provide controlled delivery of the compound and DOT1L inhibitor wherein the rate of absorption is slowed by using rate-controlling membranes or by trapping the compound and DOT1L inhibitor within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound and DOT1L inhibitor with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound and DOT1L inhibitor.

### Dosage Amounts

As used herein, the term, “therapeutically effective amount” refers to an amount of compound (1) and an amount of the DOT1L inhibitor or a pharmaceutically acceptable salt thereof that are each effective in producing the desired therapeutic response in a particular patient suffering from a cancer mediated by SMARCA4/2 and/or DOT1L protein activity. The term “therapeutically effective amount” thus includes the amount of each of the active agents or a pharmaceutically acceptable salt thereof, that when administered, induces a positive modification in the cancer to be treated, or is sufficient to prevent development or progression of the cancer, or alleviate to some extent, one or more of the symptoms of the cancer being treated in a subject, or which simply kills or inhibits the growth of cancer cells, or reduces the amounts of SMARCA4/2 and/or DOT1L protein in cancer cells.

The total daily dosage of each of the active agents and usage thereof may be decided in accordance with standard medical practice, e.g., by the attending physician using sound medical judgment. The specific therapeutically effective dose for any particular subject may depend upon a variety of factors including the cancer being treated and the severity thereof (e.g., its present status): the age, body weight, general health, sex and diet of the subject: the time of administration, route of administration, and rate of excretion of the compound employed: the duration of the treatment; drugs used in combination or coincidental with the compound; and like factors well known in the medical arts (see, for example, Goodman and Gilman's. The Pharmacological Basis of Therapeutics, 10th Edition, A. Gilman, J. Hardman and L. Limbird, eds., McGraw-Hill Press, 155-173, 2001).

Compound (1) and its pharmaceutically acceptable salts may be effective over a wide dosage range. In some embodiments, the total daily dosage (e.g., for adult humans) may range from about 0.001 to about 1600 mg, from 0.01 to about 1600 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, or in yet other embodiments from about 10 to about 30 mg per day. In some embodiments, the total daily dosage may range from 400 mg to 600 mg. Individual dosages may be formulated to contain the desired dosage amount depending upon the number of times the compound is administered per day. By way of example, capsules may be formulated with from about 1 to about 200 mg of the compound (e.g., 1, 2, 2.5, 3, 4, 5, 10, 15, 20, 25, 50, 100, 150, and 200 mg). In some embodiments, the compound may be administered at a dose in range from about 0.01 mg to about 200 mg/kg of body weight per day. In some embodiments, a dose of from 0.1 to 100, e.g., from 1 to 30 mg/kg per day in one or more dosages per day may be effective. By way of example, a suitable dose for oral administration may be in the range of 1-30 mg/kg of body weight per day, and a suitable dose for intravenous administration may be in the range of 1-10 mg/kg of body weight per day. In some embodiments, compound (1) is administered in a dose from 100 mg per day to 250 mg per day. In other embodiments, the compound is administered in a dose from 200 mg per day to 400 mg per day, e.g., 250-350 mg per day.

DOT1L inhibitors and their pharmaceutically acceptable salts may be effective over a wide dosage range. In some embodiments, the total daily dosage can range from about 0.01 mg/kg to about 5000 mg/kg. In some embodiments, dosages can range from about 1 mg/kg to about 1000 mg/kg per day. In some embodiments, the dosages can range of about 0.1 mg/day to about 50 g/day: about 0.1 mg/day to about 25 g/day: about 0.1 mg/day to about 10 g/day; about 0.1 mg to about 3 g/day: or about 0.1 mg to about 1 g/day. In some embodiments, the DOT1L inhibitor is administered in a dose from 20 mg/kg to 172 mg/kg per day. In some embodiments, the DOT1L inhibitor is administered in a dose from 56 mg/kg to 112 mg/kg per day. In other embodiments, the DOT1L inhibitor is administered in a dose 54 mg/m² to 90 mg/m² per day.

In some embodiments, compound (1) and the DOT1L inhibitor may achieve a synergistic, i.e., greater than additive effect with respect to single reagent treatment. In these embodiments, the dosage amount of compound (1) may be determined based on dosages used in the in vitro studies described in the working examples (0.03 μM to about 0.25 μM, and the DOT1L inhibitor is about 0.3 μM to about 5 μM, on a daily basis), using art-recognized models of such correlation and extant data in the literature.

### Methods of Use

An aspect of the present invention is directed to treating cancer. In some embodiments, the cancer may be characterized by expression of SMARCA4/2 and DOT1L. In some embodiments, the cancer may be characterized by expression of SMARCA4/2 and DOT1L and/or corresponding protein activity. The methods entail administering a therapeutically effective amount of compound (1) in combination with the DOT1L inhibitor or a pharmaceutically acceptable salt thereof, to a subject in need thereof.

The term “subject” (or “patient”) as used herein includes all members of the animal kingdom prone to or suffering from the indicated cancer. In some embodiments, the subject is a mammal, e.g., a human or a non-human mammal. The methods are also applicable to companion animals such as dogs and cats. A subject “in need of” treatment according to the present invention may be “suffering from or suspected of suffering from” a specific cancer may have been positively diagnosed or otherwise presents with a sufficient number of risk factors or a sufficient number or combination of signs or symptoms such that a medical professional could diagnose or suspect that the subject was suffering from the cancer. Thus, subjects suffering from, and suspected of suffering from, a specific cancer are not necessarily two distinct groups.

As used in the context of the DOT1L inhibitor, the term, “therapeutically effective amount” refers to an amount of the DOT1L inhibitor or a pharmaceutically acceptable salt thereof that is effective in producing the desired therapeutic response in a particular patient suffering from a cancer mediated by SMARCA4/2 and DOT1L activity. The term “therapeutically effective amount” thus includes the amount of the DOT1L inhibitor or a pharmaceutically acceptable salt thereof, that when administered, induces a positive modification in the cancer to be treated, or is sufficient to prevent development or progression of the cancer, or alleviate to some extent, one or more of the symptoms of the cancer being treated in a subject, or which simply kills or inhibits the growth of cancer cells, or reduces the amounts of DOT1L in cancer cells.

As used herein, the term “in combination” means that the two active agents are co-administered. The term co-administration, as used herein, includes substantially contemporaneous administration, by way of the same or separate dosage forms, and by the same or different modes of administration, or sequentially (either one before the other), e.g., as part of the same treatment regimen, or by way of successive treatment regimens. Therefore, the method is not limited to the administration of the active agents at exactly the same time. If administered sequentially, administration of the second agent is timed such that it may augment the anti-cancer effect of the first and previously administered agent. In some embodiments, the co-administration of compound (1) and the DOT1L inhibitor achieves a synergistic anti-cancer effect.

The methods of the present invention may entail administration of compound (1) and the DOT1L inhibitor, or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails a 28-day cycle which includes daily administration for 3 weeks (21 days). In other embodiments, the compounds may be dosed twice a day (BID) over the course of two and a half days (for a total of 5 doses) or once a day (QD) over the course of two days (for a total of 2 doses). In other embodiments, the compounds may be dosed once a day (QD) over the course of five days. The length of the treatment period depends on a variety of factors, such as severity of the cancer, age of the subject, the concentration and the activity of the compounds, or a combination thereof. It will also be appreciated that the effective dosage of the compounds used for the treatment may increase or decrease over the course of a particular treatment regime.

Broadly, the methods may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma and multiple myeloma. Both adult tumors/cancers and pediatric tumors/cancers are included. The cancers may be vascularized, or not yet substantially vascularized, or non-vascularized tumors.

Representative examples of cancers includes adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), cholangiocarcinoma, germ cell tumor, ovarian germ cell tumor, head and neck cancer, neuroendocrine tumors, Hodgkin's lymphoma. Ann Arbor stage III and stage IV childhood Non-Hodgkin's lymphoma. ROSI-positive refractory Non-Hodgkin's lymphoma, leukemia, lymphoma, multiple myeloma, hypopharyngeal cancer, intraocular melanoma, ocular cancer, islet cell tumors (endocrine pancreas), renal cancer (e.g., Wilm's Tumor, renal cell carcinoma), liver cancer, lung cancer (e.g., non-small cell lung cancer and small cell lung cancer), ALK-positive anaplastic large cell lymphoma, ALK-positive advanced malignant solid neoplasm, Waldenstrom's macroglobulinemia, melanoma, intraocular (eye) melanoma, Merkel cell carcinoma, mesothelioma, metastatic squamous neck cancer with occult primary, multiple endocrine neoplasia (MEN), myelodysplastic syndromes, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, metastatic anaplastic thyroid cancer, undifferentiated thyroid cancer, papillary thyroid cancer, pituitary tumor, plasma cell neoplasm/multiple myeloma, pleuropulmonary blastoma, prostate cancer, retinoblastoma, rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, juvenile xanthogranuloma, transitional cell cancer of the renal pelvis and ureter and other urinary organs, urethral cancer, gestational trophoblastic tumor, vaginal cancer, vulvar cancer, hepatoblastoma, rhabdoid tumor, and Wilms tumor.

Sarcomas that may be treatable with the methods of the present invention include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue) and mesenchymous or mixed mesodermal tumor (mixed connective tissue types), and histiocytic sarcoma (immune cancer).

In some embodiments, methods of the present invention entail treatment of subjects having cell proliferative diseases or disorders of the hematological system, liver, brain, lung, colon, pancreas, prostate, skin, ovary, breast, skin and endometrium.

As used herein, “cell proliferative diseases or disorders of the hematological system” include lymphoma, leukemia, myeloid neoplasms, mast cell neoplasms, myelodysplasia, benign monoclonal gammopathy, lymphomatoid papulosis, polycythemia vera, chronic myelocytic leukemia, agnogenic myeloid metaplasia, and essential thrombocythemia. Representative examples of hematologic cancers may thus include leukemia, multiple myeloma, and lymphoma (including T-cell lymphoma, Hodgkin's lymphoma, non-Hodgkin's lymphoma (NHL). Examples of NHL include diffuse large B-cell lymphoma (DLBCL), follicular lymphoma (FL), mantle cell lymphoma (MCL), cutaneous T-cell lymphoma (CTCL) (including mycosis fungoides and Sezary syndrome), peripheral T-cell lymphoma (PTCL) (including anaplastic large-cell lymphoma (ALCL), angioimmunoblastic T-cell lymphoma, hepatosplenic T-cell lymphoma, epithelial T-cell lymphoma, and gamma-delta T-cell lymphoma), germinal center B-cell-like diffuse large B-cell lymphoma, activated B-cell-like diffuse large B-cell lymphoma, Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, refractory NHL, relapsed NHL, childhood lymphomas, and small lymphocytic lymphoma. Examples of leukemia include childhood leukemia, hairy-cell leukemia, acute lymphocytic leukemia, acute myelocytic leukemia, acute myeloid leukemia (e.g., acute monocytic leukemia), chronic lymphocytic leukemia, chronic myelocytic leukemia, chronic myelogenous leukemia, mast cell leukemia, myeloid neoplasms and mast cell neoplasms.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), squamous cell carcinoma, adenocarcinoma, small cell carcinoma, large cell carcinoma, squamous cell carcinoma, and mesothelioma. Lung cancer can include “scar carcinoma”, bronchoalveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, the compound of the present invention may be used to treat non-metastatic or metastatic lung cancer (e.g., NSCLC, ALK-positive NSCLC, NSCLC harboring ROSI Rearrangement, Lung Adenocarcinoma, and Squamous Cell Lung Carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium.

In some embodiments, the methods treat a mixed lineage leukemia rearrangement (MLLr) cancer. In some embodiments, the MLLr cancer is leukemia. In some embodiments, the MLLr cancer is acute leukemia. In some embodiments, the leukemia is acute myeloid leukemia (AML). In some embodiments, the leukemia is acute erythroid leukemia (AEL). In some embodiments, the leukemia is acute lymphoblastic leukemia (ALL). In some embodiments, the leukemia is T-cell acute lymphoblastic leukemia (T-ALL). In some embodiments, the leukemia is adult T-cell leukemia (ATL).

### Pharmaceutical Kits

Compound (1) and its pharmaceutically acceptable salts and/or compositions containing them may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain compound (1) or a pharmaceutical composition thereof. The kits or pharmaceutical systems of the invention may also include printed instructions for using the compound and compositions.

These and other aspects of the present invention will be further appreciated upon consideration of the following Examples, which are intended to illustrate certain particular embodiments of the invention but are not intended to limit its scope, as defined by the claims.

### EXAMPLES

### Example 1: SMARCA4/2 Binding Assay

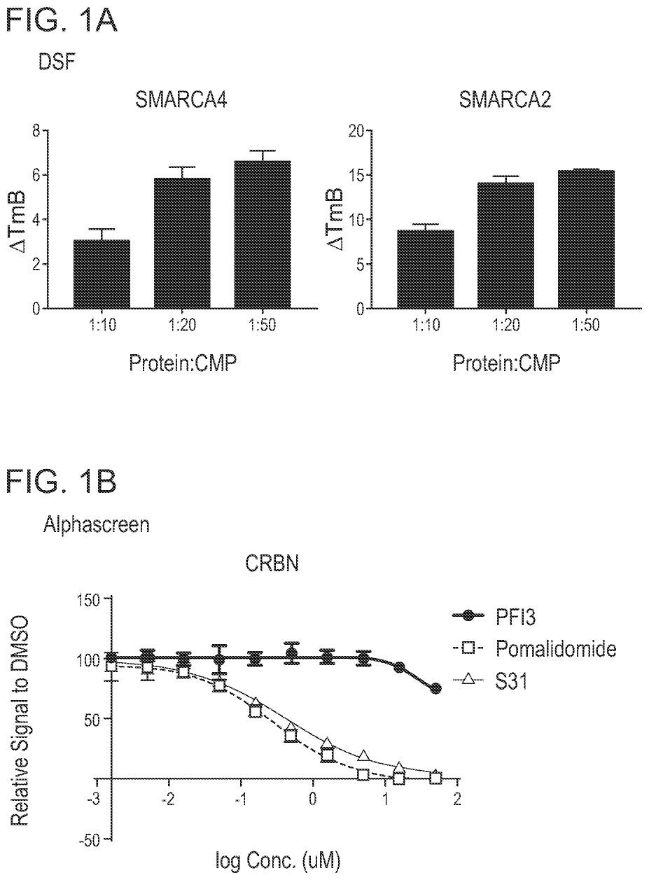

Recombinant SMARCA4 and SMARCA2 proteins were incubated with S31 at indicated ratios. After 30 min of incubation, DSF assay was performed using Protein Thermal Shift™ Dye Kit (ThermoFisher™) according to manufacture instructions (FIG. 1A).

50 nM of His-CRBN was incubated with biotinylated probe (biotin-pomalidomide), streptavidin donor beads (PerkinElmer®), nickel chelate acceptor beads (PerkinElmer®) and compounds (S31, PFI3 or pomalidomide at indicated concentrations) for 1 hr. After incubation, absorbance was read according to manufacture instructions (FIG. 1B).

### Example 2: S31 Degrades SMARCA4/2

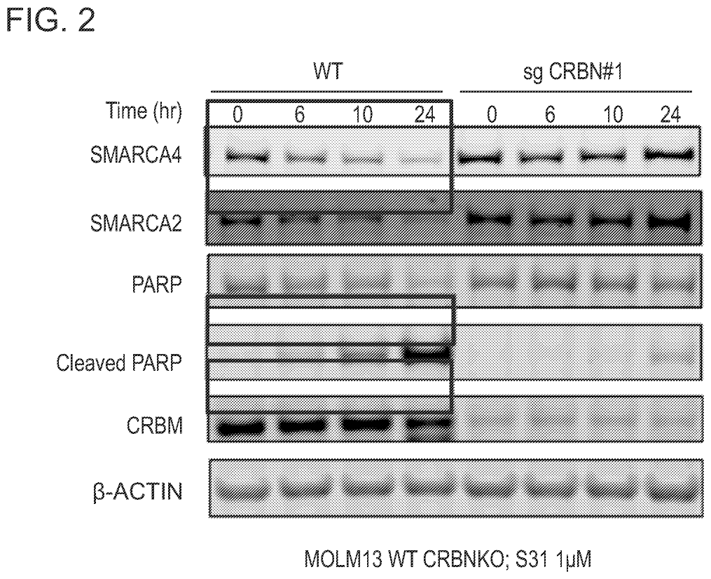

MOLM13 WT and CRBN KO cells were treated with 1 μM of S31 at indicated time points. Cell lysates were harvested, and proteins were extracted using RIPA buffer. Protein (40 μg) was loaded for Western Blotting which showed that S31 degraded SMARCA4/2 (FIG. 2).

### Example 3: S31 Induces Cytotoxicity in Leukemia Cells

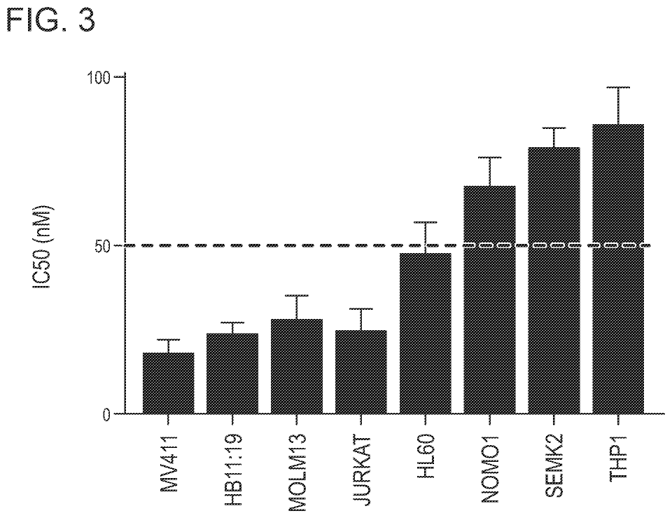

S31 was treated in a panel of leukemia cell lines with 10 doses ranging from 10 μM to 1 nM. After 72 hr of treatment, growth inhibition was determined by ATPlite™ (PerkinElmer®). IC₅₀ was calculated by GraphPad Prism® 7 (FIG. 3).

### Example 4: BRM14 and EPZ5676 Leads to Synthetic Lethality

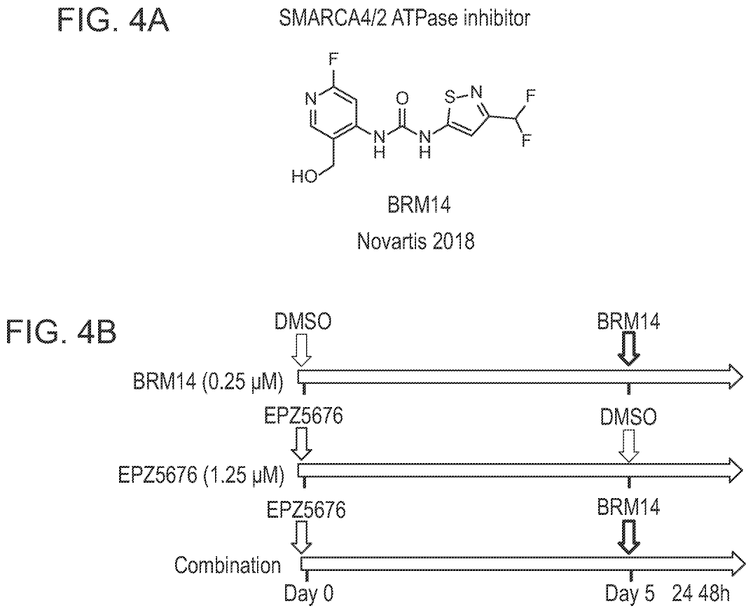

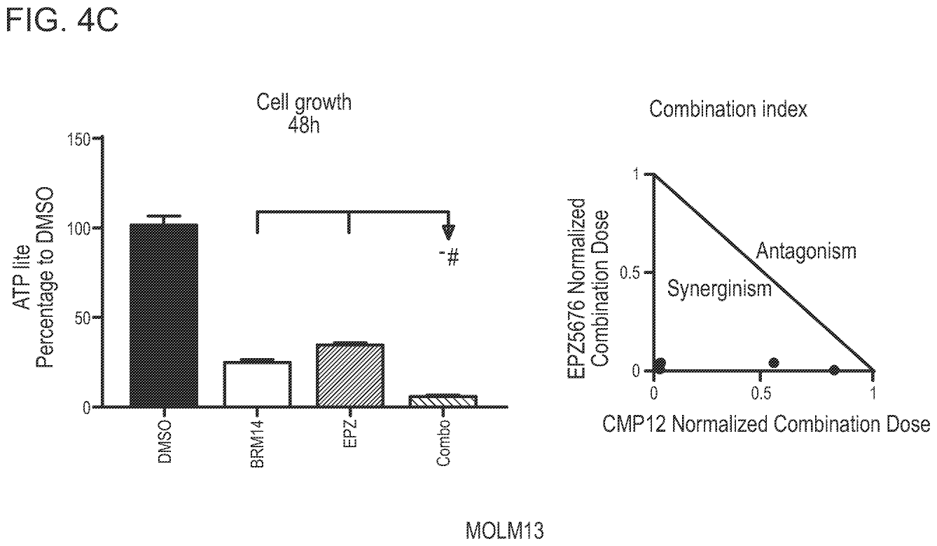

MOLM13 cells were primed with 1.25 μM of EPZ5676 or DMSO for 4 days. At day 5, additional BRM14 (0.25 μM) or DMSO was added for another 48 hr. Growth inhibition was determined by ATPlite™ (PerkinElmerR). Combination index was determined by CompuSyn software (PD Science, LLC) (FIG. 4C).

### Example 5: S31 and EPZ5676 Shows a Synergistic Effect in Treatment

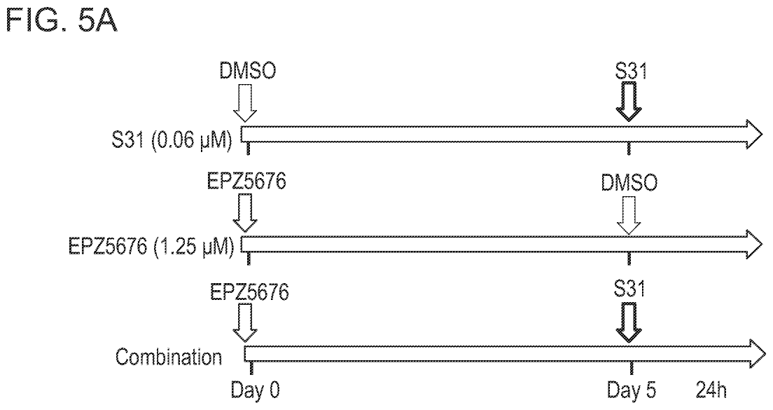

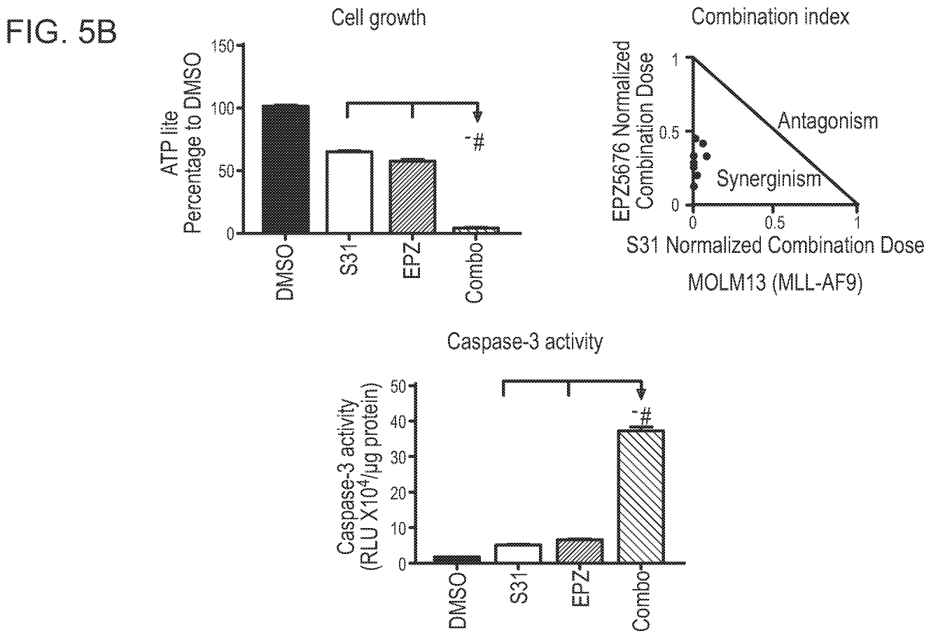

MOLM13 cells were primed with 1.25 μM of EPZ5676 or DMSO for 4 days. At day 5, additional S31 (0.06 UM) or DMSO was added for another 24 hr. Growth inhibition was determined by ATPlite™ (PerkinElmer®). Combination index was determined by CompuSyn software (PD Science, LLC). Caspase-3 activity was determined by Caspase-Glo® 3/7 Assay (Promega™) (FIG. 5B).

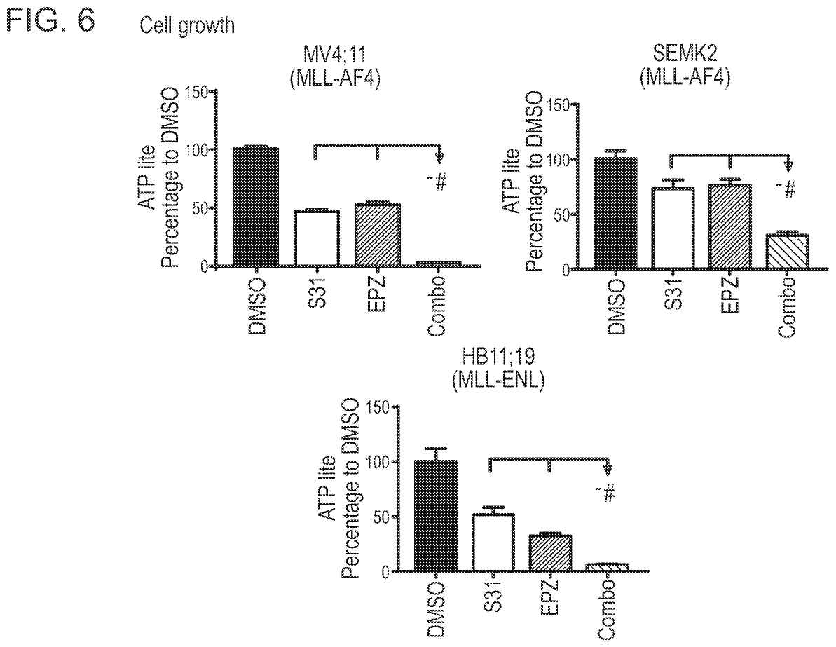

MV4; 11, SEMK2 and HB11; 19 cells were primed with 1.25 μM of EPZ5676 or DMSO for 4 days. At day 5, additional S31 (0.06 μM) or DMSO was added for another 24 hr. Growth inhibition was determined by ATPlite™ (PerkinElmer®) (FIG. 6).

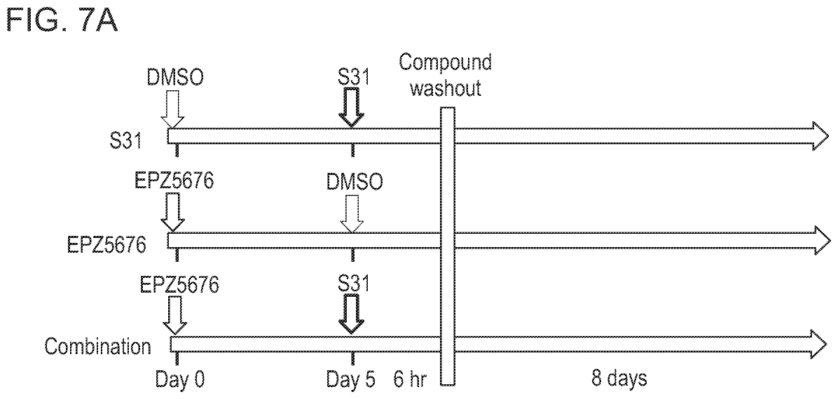

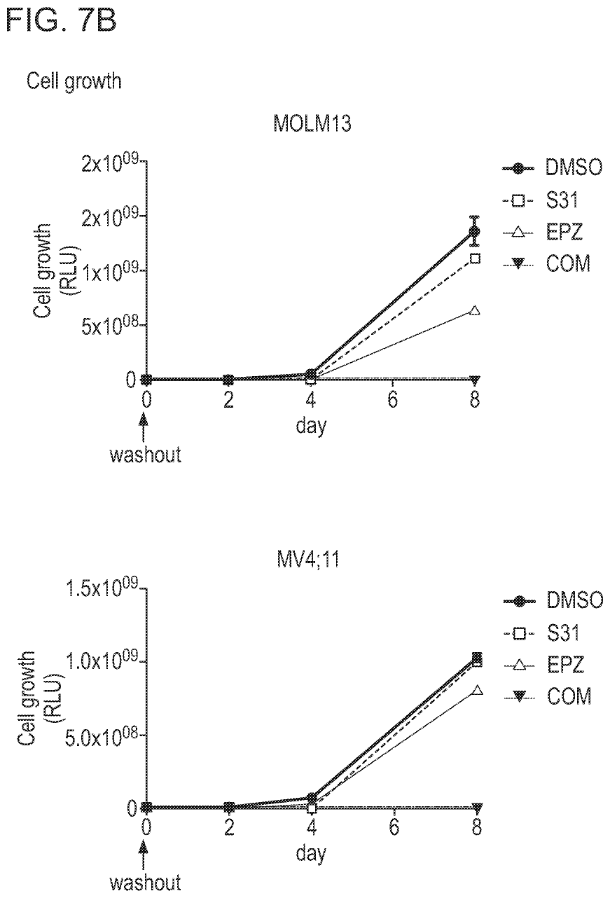

MOLM13 and MV4; 11 cells were primed with 1.25 μM of EPZ5676 or DMSO for 4 days. At day 5, additional S31 (1 μM) or DMSO was added for another 6 hr. Compounds were depleted thereafter and cells were washed with PBS (×2). Fresh growth media was replenished to keep cells growing for another 8 days. Growth inhibition was determined by ATPlite™ (PerkinElmer®) (FIG. 7B).

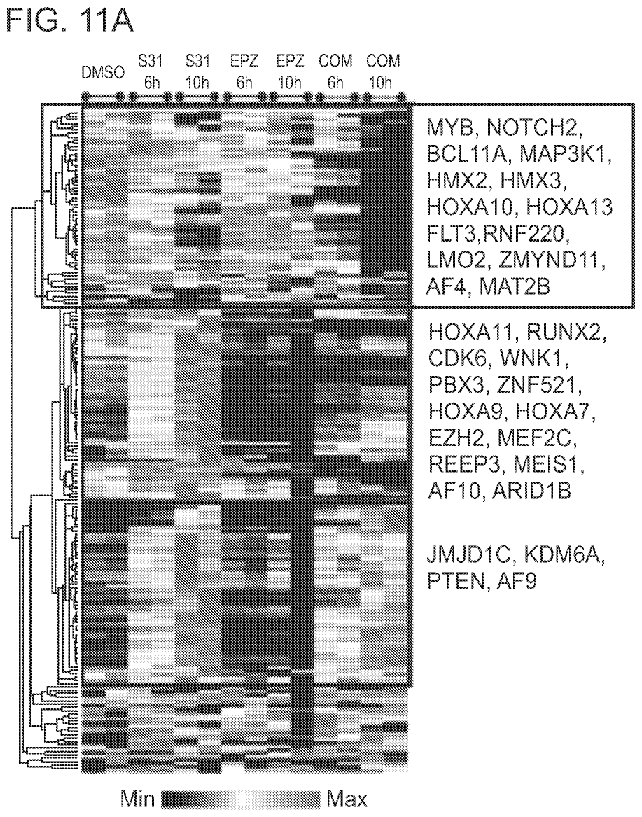

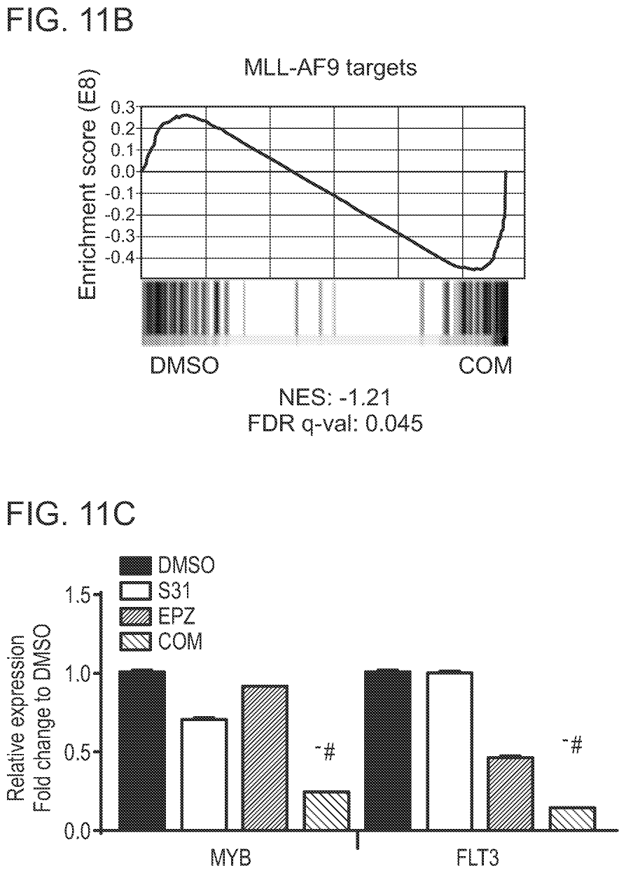

MOLM13 cells were primed with 1.25 μM of EPZ5676 or DMSO for 4 days. At day 5, additional S31 (1 μM) or DMSO was added for another 6-10 hr. Cells were harvested and subjected to RNA isolation. Isolated RNA was sent to Novogene (USA) for RNA sequencing. Expression values of MLL-AF9 targets from each treatment group were plotted in heatmap (Morpheus) (FIG. 11A). GSEA analysis was performed by GSEA software 4.1.0 (Broad Institute) (FIG. 11B).

MOLM13 cells were primed with 1.25 μM of EPZ5676 or DMSO for 4 days. At day 5, additional S31 (1 μM) or DMSO was added for another 6-10 hr. Cells were harvested and subjected to RNA isolation. QPCR of MYB and FLT3 was performed thereafter (FIG. 11C).

### Example 6: Protocol for Evaluating Efficacy of S31 and EPZ5676 Ex Vivo

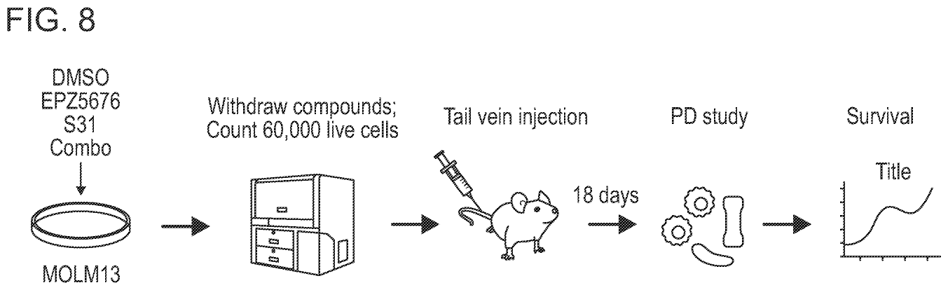

MOLM13 cells were primed with 1.25 μM of EPZ5676 or DMSO for 4 days. At day 5, additional S31 (1 μM) or DMSO was added for another 6 hr. Compounds were depleted thereafter and cells were washed with PBS (×2). 60,000 live MOLM13 cells of each treatment group were counted by flow cytometry and injected to NSG mice through tail vein (n=7/treatment). After 18 days, 3 animals were scarified, and bone marrow was collected for PD study. The rest of animals were housed for survival study (FIG. 8).

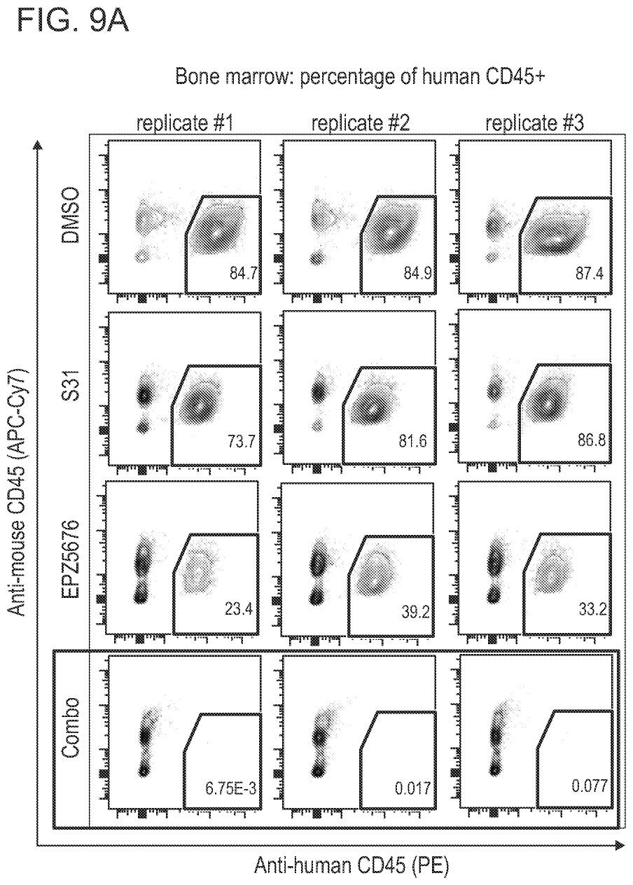

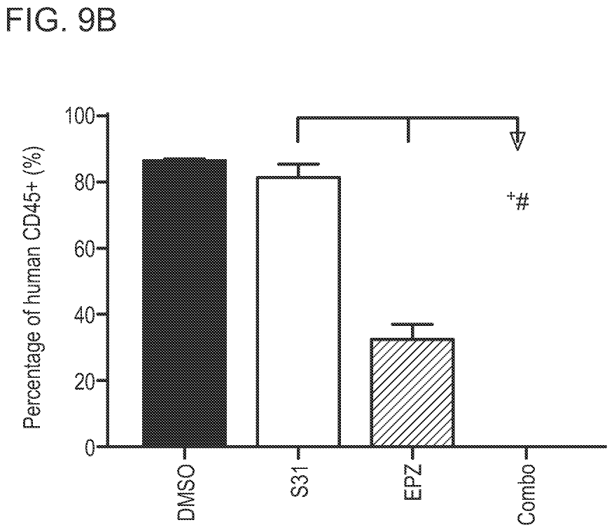

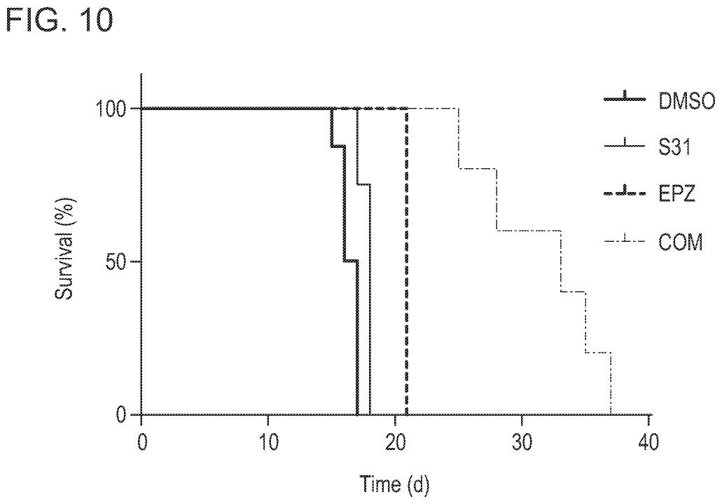

Bone marrow cells were isolated and stained with anti-human CD45 (PE) and anti-mouse CD45 (APC-Cy7) antibodies. Flow cytometry was performed to differentiate human vs. mouse CD45 cell population (FIG. 9A) and quantified (FIG. 9B). A survival curve was plotted using GraphPad Prism® 7 (FIG. 10).

### Example 7: S31 and CMP10 Shows a Synergistic Effect in Treatment

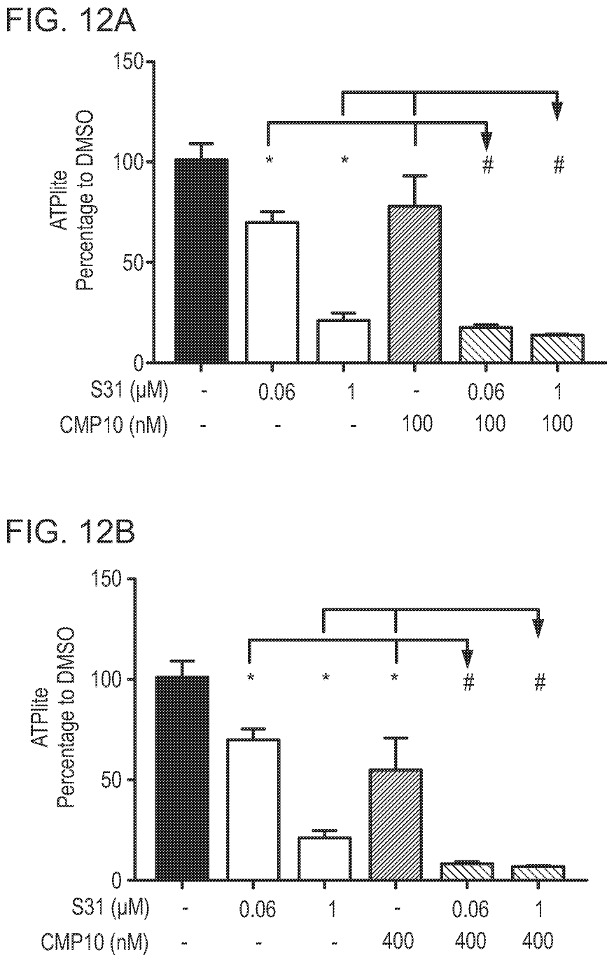

MOLM13 cells were primed with 100 nM or 400 nM CMP10 or DMSO for 4 days. At day 5, S31 (0.6 or 1 μM) or DMSO was added for another 24 hr. Growth inhibition was determined by ATPlite™ (PerkinElmer®) (FIG. 12A and FIG. 12B).

### Example 8: S31 and CMP11 Shows a Synergistic Effect in Treatment

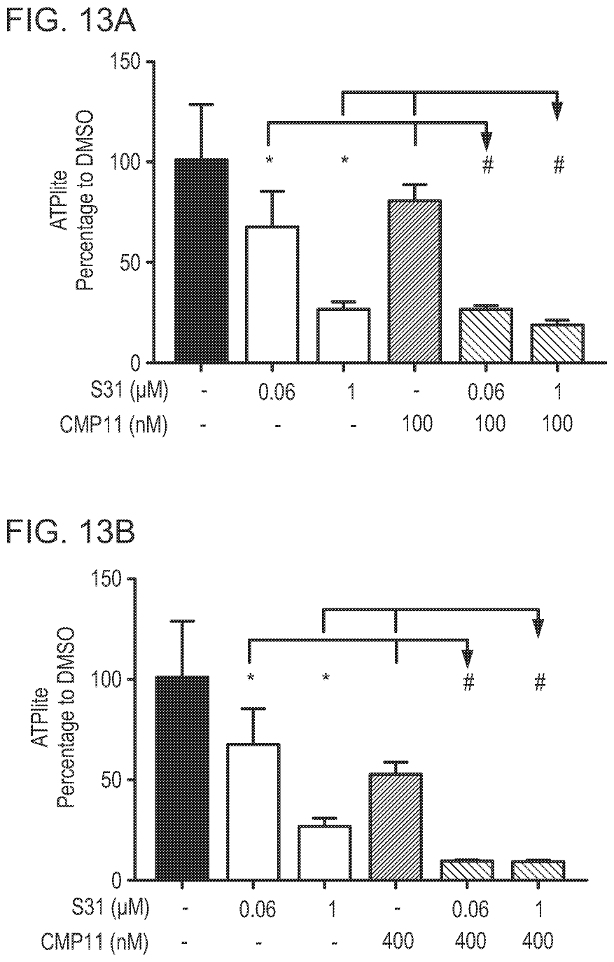

MOLM13 cells were primed with 100 nM or 400 nM CMP11 or DMSO for 4 days. At day 5, S31 (0.6 or 1 μM) or DMSO was added for another 24 hr. Growth inhibition was determined by ATPlite™ (PerkinElmer®) (FIG. 13A and FIG. 13B).

### Summary of Results

Since open and accessible chromatin facilitates the transcription of MLL fusion target genes, the disruption of chromatin remodeling complex function should enhance the interruption of DOT1L function and provide mechanistic insight for MLLr. SWI/SNF complex is the most commonly mutated ATP-dependent chromatin remodeling complexes, with a collective frequency of approximately 20% across various cancers. SWI/SNF complexes utilize energy derived from ATP hydrolysis to disrupt histone-DNA contacts, and thereby allow access of transcription factors to their cognate DNA elements. SMARCA4 as well as its homologue, SMARCA2, is the conserved catalytic ATPase subunit in all SWI/SNF subcomplexes. Interestingly, SMARCA4 is identified as an essential supporter of the oncogenic transcriptional programs and acts as one of the top chromatin regulator dependencies in MLLr acute leukemia. The functional outcome and molecular basis of targeting both DOT1L and SMARCA4/2 in MLLr leukemia were studied.

Chemical inhibition of targeted proteins offered the opportunity to understand the function and therapeutic potential of the targets with temporal control. There are several small molecule inhibitors that have been reported to target SMARCA2/4. The inhibitors developed to target specific domains or components of the complexes showed limited overall effects, which limited their usage to understand SWI/SNF function. Therefore, PROTACs, bridging small molecules that recruit the target protein into proximity with the E3 ligase system to initiate proteasomal degradation of targeted proteins inside the cells, were developed to target SMARCA2/4. The strategy has recently been described and utilized for the depletion of multiple targets including BRD4, FKBP12, ERR, RIPK2 and BRD9. Compound (1) (S31) is a molecule that contains phthalimide (thalidomide, lenalidomide and pomalidomide, also known as IMiDs®), a CRBN binder, and a SMARCA4/2 binder that are linked via an alkyl linker. The PROTAC dimerized targeted proteins and CRBN, and induced polyubiquitylation followed by proteasomal degradation of SMARCA4/2. Using the potent and selective SMARCA4/2 degrader (S31), both SMARCA4 and SMARCA2 degradation was observed and the cleaved PARP expression abundance was observed after 6 hours of treatment, which indicated apoptosis. The effects of S31 on cell growth of a panel of leukemia cell lines was tested and profound inhibition was observed in cell growth with IC₅₀ of low nanomolar range.

The combination treatment of DOT1L inhibition and SMARCA4/2 inactivation was evaluated in MLLr leukemia. As downregulation of H3K79 methylation by EPZ5676 required relatively long-time exposure, the combinatorial assay started with exposure of cells to EPZ5676 or DMSO for 4 consecutive days. SMARCA4/2 degrader (S31) or inhibitor (BRM14) was then added on day 5 to induce SMARCA4/2 inactivation. While the single agents manifested certain extent of inhibition in cell growth, the combination of agents demonstrated significantly greater reduction of cell numbers in MOLM13 cells. The comparison of results after treatment with various doses of S31 or BRM14 and/or EPZ5676 unveiled a synthetic lethality determined by combination index (CI) analysis. Furthermore, caspase-3 activity assay indicated that SMARCA4/2 inactivation together with DOT1L inhibition enhanced apoptosis. The cooperativity between SMARCA4/2 and DOT1L was determined in other MLLr leukemia cells and similar synthetic lethality was observed. More importantly, the synthetic lethality generated by S31+EPZ5676 was long-lasting even after compound withdrawal. This effect was evaluated in an ex vivo mouse model by injecting the compound treated cancer cells into NSG mice. The combination treatment dramatically suppressed leukemia cell expansion in bone marrow and provided a dramatic survival benefit. Thus, the data suggests that inactivation of SMARCA4/2, either by blocking its catalytic function or degrading the protein, generated an enhanced anti-tumor activity when combined with EPZ5676. This combination modality provides a method for the treatment of MLLr AML.

The molecular interdependency between DOT1L inhibition and SMARCA4/2 inactivation was explored using RNA-seq in MOLM13 cells treated with DMSO. S31. EPZ5676 and S31+EPZ5676. Persistent expression of MLL fusion targets maintained the leukemic transformation as a stem cell-like state. Genes changed by combination treatment were significantly correlated with genes directly bound by MLL-AF9. To define a detailed regulatory pattern of the effects of SMARCA4/2 and DOT1L on these genes, the RNA-seq results were plotted in a heatmap and identified one subset of genes are of particular interest. In this subset, EPZ5676 or S31 could not—or just slightly—affect the gene expression. However, the combination of the two induced significant reduction of genes such as MYB and FLT3, which are critical for MLLr leukemia proliferation. S31 sensitized such MLL-AF9) targets to EPZ5676. This was confirmed via Q-PCR.

The development of the combination strategy together with translational validation produce insights of dual targeting MLL complex and chromatin remodeling complex as cancer therapeutic strategy. The translational nature of compound 1 fully assess the SMARCA4/2 inactivation and DOT1L inhibition in both cancer cells and in animal model to establish the pre-clinical rationale. Moreover, the tools are valuable to cancer research community to fully uncover deep mechanistic insights of MLLr driven leukemia. In addition, the benefit of the small molecule allows for further understanding of SNI/SWF complex functions in varieties of cancers and the fundamental basic biological understanding for MLLr leukemias.

The ex vivo xenograft model was used to evaluate the efficacy and fully assess the anti-tumor effects of the disclosed combination strategy. The NOD-SCID-IL2Rnull (NSG) mice were orthotopically xenografted with MV4:11 or MOLM13 cells via injection by the lateral tail vein. The cells were treated with single agent, combination, or DMSO control before the implantation. Each of the 4 treatment arms (vehicle control, EPZ5676, S31, and EPZ5676+S31) used 9 mice, 3 in short-term mechanistic studies and 6 in efficacy studies (FIG. 8). Two weeks after inoculation, the blood samples were collected from mice, and FACS with hCD45+ mark was used to check the tumor burden (FIG. 9A and FIG. 9B). When the mouse developed clinical symptoms, such as weakness or paralysis, the mice were sacrificed, and the blood sample as well as liver and spleen were collected to confirm the tumor burden with human CD45+ antibodies. The animal survival days were used to measure the survival benefit (FIG. 10). This further established the pre-clinical rationale for the combination strategy in leukemia.

To elucidate the mechanistic basis of synthetic lethality induced by dual targeting DOT1L and SMARCA4/2, transcriptional profiling was performed by RNA-seq. In parallel, the findings were validated in SMARCA4/2 and/or DOT1L genetic knockout/knockdown systems. These data identified a group of MLL-target genes that was controlled specifically by combination treatment (FIG. 11A and FIG. 11B).

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A method of treating cancer, comprising co-administering a therapeutically effective amount of compound (1): 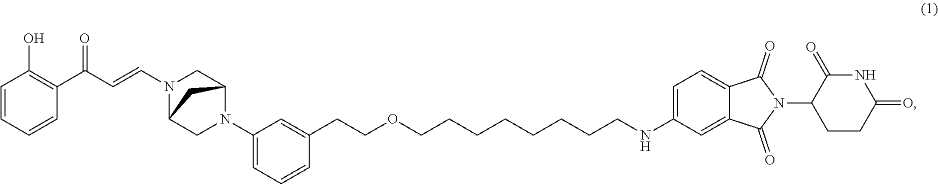 [SMILES: O=C1CCC(N2C(=O)c3ccc(NCCCCCCCCOCCc4cccc(N5C[C@H]6C[C@@H]5CN6/C=C/C(=O)c5ccccc5O)c4)cc3C2=O)C(=O)N1]
or a pharmaceutically acceptable salt thereof, and a therapeutically effective amount of a DOT1L inhibitor, or a pharmaceutically acceptable salt thereof, to a subject in need thereof.

2. The method of claim 1, wherein the DOT1L inhibitor is EPZ-5676.

3. The method of claim 1, wherein the DOT1L inhibitor is Dot1L-IN-4.

4. The method of claim 1, wherein the DOT1L inhibitor is Dot1L-IN-5.

5. The method of claim 1, wherein the cancer is a mixed lineage leukemia rearrangement (MLLr) cancer.

6. The method of claim 5, wherein the MLLr cancer is leukemia.

7. The method of claim 6, wherein the leukemia is acute myeloid leukemia (AML), acute erythroid leukemia (AEL), acute lymphoblastic leukemia (ALL), T-cell acute lymphoblastic leukemia (T-ALL), or adult T-cell leukemia (ATL).

8. The method of claim 7, wherein the leukemia is AML.

9. The method of claim 1, comprising co-administering a therapeutically effective amount of compound (1): 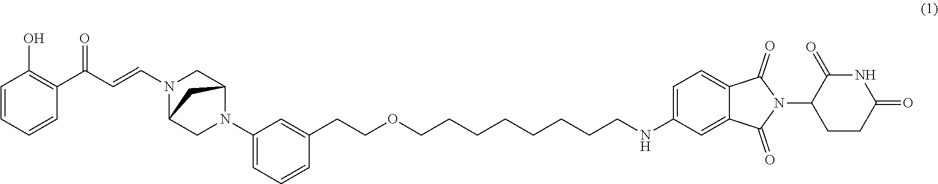 [SMILES: O=C1CCC(N2C(=O)c3ccc(NCCCCCCCCOCCc4cccc(N5C[C@H]6C[C@@H]5CN6/C=C/C(=O)c5ccccc5O)c4)cc3C2=O)C(=O)N1]
and a therapeutically effective amount of a DOT1L inhibitor, or a pharmaceutically acceptable salt thereof, to a subject in need thereof.

10. The method of claim 2, wherein the cancer is a MLLr cancer.

11. The method of claim 3, wherein the cancer is a MLLr cancer.

12. The method of claim 4, wherein the cancer is a MLLr cancer.
